(19) United States
(12) Patent Application Publication
(10) Pub. No.: US 2020/0197125 A1
(43) Pub. Date: Jun. 25, 2020

(54) HOSPITAL BED WITH ATTACHED CAREGIVER SEATING SYSTEMS

(71) Applicant: UNIVERSITY HOSPITALS HEALTH SYSTEM, INC., Cleveland, OH (US)

(72) Inventors: Cheryl O'MALLEY, Cleveland, OH (US); Douglas J. PAIGE, Cleveland, OH (US); Justin G. HOBBS, Cleveland, OH (US); Yiping MA, Cleveland, OH (US); Mindy M. ROLINCE, Cleveland, OH (US); Ginikanwa U. UZEGBU, Cleveland, OH (US); Jingde ZHANG, Cleveland, OH (US)

(21) Appl. No.: 16/612,602

(22) PCT Filed: May 31, 2018
(86) PCT No.: PCT/US2018/035293

§ 371 Date: Nov. 11, 2019

Related U.S. Application Data
(60) Provisional application No. 62/515,202, filed on Jun. 5, 2017.

(51) Int. Cl. A61B 90/60; A61G 7/05

(52) U.S. Cl. CPC A61B 90/60; A61G 7/0524

(57) ABSTRACT

A hospital bed includes a bed portion, a frame supporting the bed portion, and a seating system connected to the frame and including a seat component. The seating system is moveable between a stored state in which the seat component is at least parrtially located within an expanse of the frame, and a seating position in which the seat component is spaced apart from the frame for seating on the seat component. The seating system may include a rotating joint and a lateral bar that extends from the joint to a seat support so the seat component can be rotated away from the frame, in either a single pivot configuration, or double pivot configuration with multiple lateral bar segments and rotating joints. The seating system may include a flip seat design in which a seat component is moved out of the stored state and rotated to the seating position.

### RELATED APPLICATIONS

This application claims the benefit of U.S. Provisional Application No. 62/515,202 filed Jun. 5, 2017, which is incorporated herein by reference.

### TECHNICAL FIELD OF THE INVENTION

The technology of the present disclosure relates generally to hospital bed configurations, and more specifically to hospital bed configurations including enhanced caregiver seating systems to improve the patient experience.

### BACKGROUND

An in-patient hospital stay can be an intimidating experience. The patient experience can be improved by enhancing the doctor-patient interaction, which can be difficult within the confines of a typical hospital room. When a patient is lying in a hospital bed, having the physician, nurse, or other caregiver standing bedside can be an uncomfortable experience. Patients have reported that a feeling of the caregiver “standing over” the patient can cause discomfort and be a bit intimidating. This discomfort is more than tangential to care, as any interference with a positive patient experience with the caregiver can reduce the quality of care.

The discomfort can be reduced by having the caregiver sit next to the patient rather than stand bedside. Sitting places the patient and physician or other caregiver at a more equal level, which reduces the feeling of intimidation and the level of potential associated discomfort. Conventionally, a simple solution has been simply to place a chair in the hospital room next to the bed. This solution, however, often is inadequate within the confines of a typical hospital room. The hospital bed, space for medical equipment, shelving, drawers, and other containers for patient items and medical supplies, and the like can render an additional chair or other seat more of an obstacle than a benefit. Accordingly, current hospital room configurations have not adequately improved the patient experience in a manner that is suitable within the confines of a typical hospital room.

### SUMMARY

In view of the deficiencies of conventional hospital beds as they are accommodated in a typical hospital room, the present invention improves over conventional configurations by providing a hospital bed incorporating in combination an attached caregiver seating system. The seating system is repositionable from a stored state to a seating position. In the stored state, the seating system takes up minimal or no space within the hospital room beyond the bed portion of the combination, and the seating system is readily moved from the stored state to the seating position to permit a caregiver to sit beside. In this manner, when in the seating position the attached seating system provides a more face-to-face, eye level interaction at a comfortable person-to-person spacing, and the stored state prevents the seating system from otherwise being an obstacle within the confines of the hospital room.

An aspect of the invention is a bed including a bed portion, a frame that supports the bed portion, and an enhanced a seating system that is connected to a component of the frame, the seating system including a seat component. The seating system is moveable between a stored state in which the seat component is at least partially located within an expanse of the frame, and a seating position in which the seat component is spaced apart from the frame thereby permitting a user to sit on the seat component.

In an exemplary embodiment, the frame includes a longitudinal support that runs along a length of the bed, and the seating system may include a track that is fixed to the longitudinal support, a clamp that is slidably fixed to the track, and a lateral bar that extends from the clamp to a seat support that supports the seat, and the connection of the lateral bar to the clamp forms a rotating joint. The seating system is moveable between the stored state and the seating position by sliding the clamp along the track and rotating the lateral bar relative to the clamp via the rotating joint.

In an exemplary embodiment, the frame includes a longitudinal support that runs along a length of the bed and a cross support that is attached perpendicularly to the longitudinal support, the cross support further defining a hollowed recess. The seating system may include a rotating joint, a lateral bar, and a seat support that supports the seat, the lateral bar extending between the rotating joint at a first end and the seat support at an opposite second end. When the seating system in in the stored state the rotating joint and a portion of the lateral bar are located within the hollowed recess, and the seating system is moveable between the stored state and the seating position by pulling the lateral bar out from the hollowed recess and rotating the lateral bar via the rotating joint.

In an exemplary embodiment, the frame includes a longitudinal support that runs along a length of the bed, and the seating system may include a joint structure that is fixed to the longitudinal support, and a lateral bar that extends from the joint structure to a seat support that supports the seat, and the connection of the lateral bar to the joint structure forms a rotating joint. The seating system is moveable between the stored state and the seating position by rotating the lateral bar relative to the longitudinal support via the rotating joint. The joint structure may include a first joint connected to the longitudinal support, the first joint permitting rotation of the lateral bar relative to the longitudinal support, and a second joint connected to the first joint and the lateral bar, the second joint permitting a vertical movement of the lateral bar between a lower position for seating and an upper position for the stored state.

In an exemplary embodiment, the frame includes a longitudinal support that runs along a length of the bed. The seating system may include a lateral bar divided into segments, a segment of the lateral bar being connected to a seat support that supports the seat, and multiple rotating joint components that connect adjacent segments of the lateral bar and that connect the lateral bar to the longitudinal support, thereby forming a double pivot mechanism. The seating system is moveable between the stored state and the seating position by rotating the lateral bar relative to the longitudinal support and rotating segments of the lateral bar relative to each other via the rotating joints. The seating system may include a first rotating joint that is connected to the longitudinal support, a first lateral bar component that is connected to the first rotating joint at a first end, a second rotating joint that is connected to a second end of the first lateral bar component, and a second lateral bar component that is connected to the second rotating joint at a first end and the seat support at a second end. The seating system is moveable between the stored state and the seating position by rotating the first lateral bar component relative to the longitudinal support via the first rotating joint, and rotating the second lateral bar component relative to the first lateral bar component via the second rotating joint.

In an exemplary embodiment, the seating system includes a bedrail attached to the frame and that includes a seat housing, and the seat component is rotatable relative to the seat housing. In the stored state the seat component is located substantially within the seat housing, and the seating system is moveable from the stored state to the seating position by moving the seat component out from the seat housing and rotating the seat component relative to the seat housing.

In an exemplary embodiment, the seating system includes one or more bedrails attached to the frame, a pivotal clamp that is attached to the frame, and the seat component is connected to the pivotable clamp and is rotatable via the pivotable clamp. In the stored state the seat component is located adjacent to the one or more bedrails, and the seating system is moveable from the stored state to the seating position by moving the seat component upward relative to the frame and rotating the seat component via the pivotable clamp.

These and further features of the present invention will be apparent with reference to the following description and attached drawings. In the description and drawings, particular embodiments of the invention have been disclosed in detail as being indicative of some of the ways in which the principles of the invention may be employed, but it is understood that the invention is not limited correspondingly in scope. Rather, the invention includes all changes, modifications and equivalents coming within the spirit and terms of the claims appended hereto. Features that are described and/or illustrated with respect to one embodiment may be used in the same way or in a similar way in one or more other embodiments and/or in combination with or instead of the features of the other embodiments.

### BRIEF DESCRIPTION OF THE DRAWINGS

FIG. 1 is a drawing depicting a conventional hospital bed and the outline of a patient.

FIG. 2 is a drawing depicting a first exemplary hospital bed in accordance with embodiments of the present invention, having a first exemplary seating system shown in a stored position.

FIG. 3 is a drawing depicting various views of the first exemplary seating system.

FIG. 4 is a drawing depicting a perspective view of the first exemplary seating system.

FIG. 5 is a drawing depicting a closeup view of track and clamp portions of the first exemplary seating system.

FIG. 6 is a drawing depicting another closeup view of the track and clamp portions of the first exemplary seating system.

FIG. 7 is a drawing depicting movement of the first exemplary seating system from the stored state of FIG. 2 to a seating position.

FIG. 8 is a drawing depicting a second exemplary hospital bed in accordance with embodiments of the present invention, having a second exemplary seating system shown in a stored position.

FIG. 9 is a drawing depicting a plan view of the second exemplary hospital bed.

FIG. 10 is a drawing depicting a first step in operation of the second exemplary seating system.

FIG. 11 is a drawing depicting a second step in operation of the second exemplary seating system.

FIG. 12 is a drawing depicting a third step in operation of the second exemplary seating system.

FIG. 13 is a drawing depicting movement of the second exemplary seating system from the stored state of FIG. 8 to a seating position.

FIG. 14 is a drawing depicting a third exemplary hospital bed in accordance with embodiments of the present invention, having a third exemplary seating system shown in a stored position.

FIG. 15 is a drawing depicting a closeup view of a joint structure of the third exemplary seating system.

FIG. 16 is a drawing depicting another closeup view of the joint structure of the third exemplary seating system.

FIG. 17 is a drawing depicting a first step in operation of the third exemplary seating system.

FIG. 18 is a drawing depicting a second step in operation of the third exemplary seating system.

FIG. 19 is a drawing depicting a third step in operation of the third exemplary seating system.

FIG. 20 is a drawing depicting movement of the third exemplary seating system from the stored state of FIG. 14 to a seating position.

FIG. 21 is a drawing depicting a fourth exemplary hospital bed in accordance with embodiments of the present invention, having a fourth exemplary seating system shown in a stored position.

FIG. 22 is a drawing depicting a side view of the hospital bed of FIG. 21.

FIG. 23 is a drawing depicting a top view of the hospital bed of FIG. 21.

FIG. 24 is a drawing depicting a first step in operation of the fourth exemplary seating system.

FIG. 25 is a drawing depicting a second step in operation of the fourth exemplary seating system.

FIG. 26 is a drawing depicting a third step in operation of the fourth exemplary seating system.

FIG. 27 is a drawing depicting a range of positioning of the fourth exemplary seating system from a perspective view.

FIG. 28 is a drawing depicting a range of positioning of the fourth exemplary seating system from a plan view.

FIG. 29 is a drawing depicting a fifth exemplary hospital bed in accordance with embodiments of the present invention, having a fifth exemplary seating system shown in a seating position.

FIG. 30 is a drawing depicting a portion of the hospital bed of FIG. 29 with the fifth exemplary seating system in the stored state.

FIG. 31 is a drawing depicting the fifth exemplary seating system isolated from the other bed components and showing the seating system in the stored state.

FIG. 32 is a drawing depicting the fifth exemplary seating system isolated from the other bed components and showing the seating system in the seating position.

FIG. 33 is a drawing depicting operation of the fifth exemplary seating system.

FIG. 34 is a drawing depicting a closeup view of a portion of the fifth exemplary seating system with the seat portion in the stored state.

FIG. 35 is a drawing depicting another closeup view of a portion of the fifth exemplary seating system that illustrates the mechanical connections and interface of the seat housing and seat portion.

FIG. 36 is a drawing depicting positioning of the fifth exemplary seating system.

FIG. 37 is a drawing depicting a sixth exemplary hospital bed in accordance with embodiments of the present invention, having a sixth exemplary seating system shown in a seating position.

FIG. 38 is a drawing depicting the hospital bed of FIG. 37 with the sixth exemplary seating system in the stored state.

FIG. 39 is a drawing depicting operation of the sixth exemplary seating system.

FIG. 40 is a drawing depicting a closeup view of a portion of the sixth exemplary seating system with the seat portion removed and showing a pivotable clamp.

FIG. 41 is a drawing depicting a perspective view of the seating portion of the sixth exemplary seating system in isolation.

FIG. 42 is a drawing depicting positioning of the sixth exemplary seating system.

### DETAILED DESCRIPTION OF EMBODIMENTS

Embodiments will now be described with reference to the drawings, wherein like reference numerals are used to refer to like elements throughout. It will be understood that the figures are not necessarily to scale.

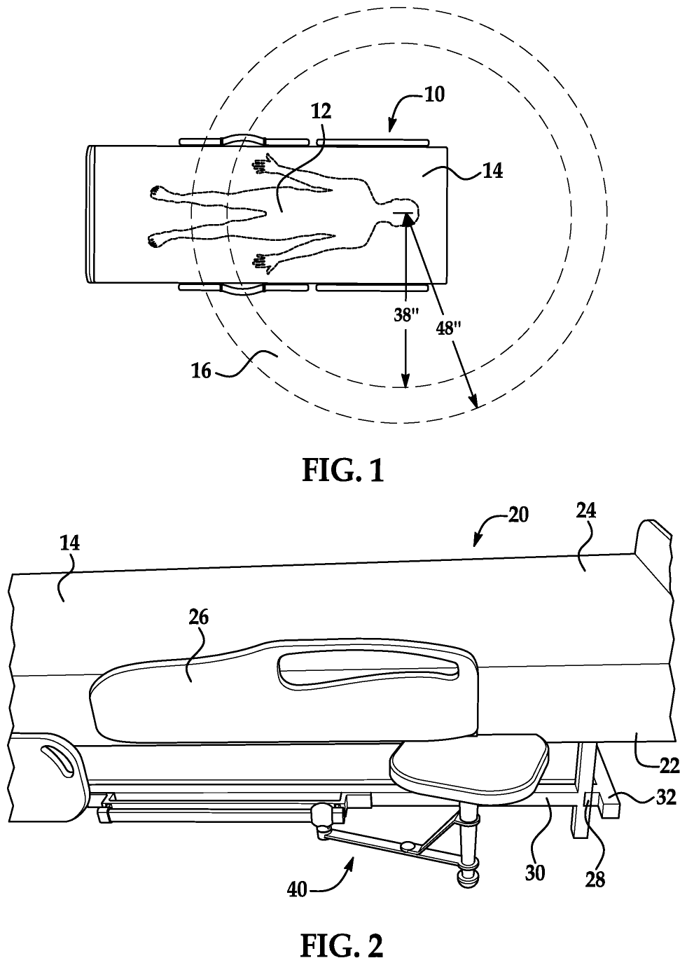

FIG. 1 is a drawing depicting a conventional hospital bed 10 and the outline of a patient 12. Applicant has researched what positioning of a caregiver relative to the patient provides an optimal patient experience. As referenced above, it is desirable that the physician or other caregiver be sitting bedside such that the patient and caregiver are at a more equal level, which reduces the feeling of intimidation and the level of potential associated discomfort. It further is desirable for the caregiver to be able to pivot, for example to turn to speak to family members or others in the room (e.g., nurses, other physicians). In addition to being at eye level, distance from the patient's eyes is significant for maintaining a comfortable person-to-person spacing between the caregiver and patient.

Accordingly, the inventors have used patient interviews, surveys and related information gathering tools to research optimal positioning of the caregiver relative to the patient, and particularly relative to a head portion 14 of the bed 10 where the patient's head typically would lie. Based on such research, the inventors have found that at essentially patient eye level or approximately eyelevel, there is a “zone of comfort” relative to the head portion 14 of the bed that maximizes the comfort level of the patient during interactions with the caregiver. This zone of comfort is indicated in FIG. 1 as element 16. In particular, the inventors have found that maximum patient comfort is achieved when the caregiver is approximately at eyelevel with the patient, and positioned in a circumferential zone relative to the patient's eyes having radius between about 38″ to 48″. With reference to being approximately at eyelevel, there is a comfort range of about plus or minus twenty-four inches vertically relative to precisely eyelevel. In exemplary embodiments of the disclosed seating systems, therefore, one feature is to configure the seating systems to be positionable in a seating position within the zone of comfort 16 with the caregiver being seated between about 38″ to 48″ from the typical head portion 14 of the bed 10.

FIGS. 2-7 are drawings depicting a first exemplary hospital bed 20 in accordance with embodiments of the present invention. Turning initially to FIG. 2, for example, the hospital bed 20 includes an upper frame 22 that supports a bed portion 24 upon which the patient lies. The bed portion 24 typically is a mattress that is housed within or supported by the upper frame 22. The upper frame 22 further may support one or more bedrails 26 that can act as guards to prevent the patient from slipping out of the bed (such as when the patient is sleeping), and can be used to aid the patient in getting into and out of the bed. The hospital bed 20 further may include a lower frame 28. The lower frame 28 may include a plurality of rigid supports, such as metal supports, which provide a support structure for the bed components as a whole. For example, the lower frame 28 may include longitudinal supports 30 that run along the bed length, and cross supports 32 that run perpendicularly between the longitudinal supports 30 along the bed width. In this first example, the hospital bed 20 may include a seating system 40 that is part of the hospital bed 20. In the particular example of FIG. 2, the seating system 40 is attached to the lower frame 28, and more specifically is attached to one of the longitudinal supports 30 of the lower frame 28.

FIG. 2 depicts the seating system 40 in a stored state. In such stored state, the seating system is positioned such that a seat component of the seating system is located at least partially within the expanse of the bed frame, and particularly positioned at least partially between upper frame 22 and lower frame 28 in this example. Accordingly, in the stored state there is minimal intrusion of the seating system 40 into the hospital room.

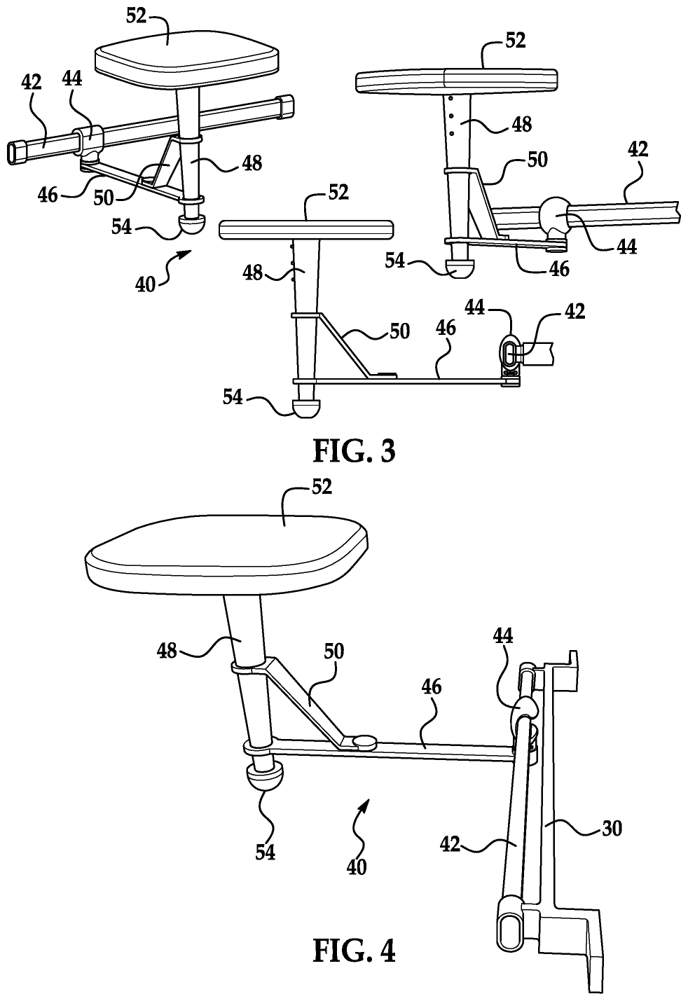

FIGS. 3 and 4 are drawings depicting various views of the seating system 40 in accordance with embodiments of the present invention. The seating system 40 may include a track 42 that is fixed to the lower frame 28 via one of the longitudinal supports 30. The track 42 may be a metal track that is welded or otherwise secured to the longitudinal support 30. The seating system 40 further may include a clamp 44 that is slidably fixed to the track 42. A lateral bar 46 may extend from the clamp 44 at a first end to a seat support 48 at a second end of the lateral bar opposite from the first end. The connection of the lateral bar 46 and seat support 48 may be reinforced using a reinforcement bar 50 that extends in a sloped configuration between the lateral bar and the seat support. The track, lateral bar, seat support, and reinforcement bar may be made of any suitable rigid material, such as a rigid plastic or metal. Aluminum components are suitable to provide adequate strength with reduced weight relative to other metal materials.

A top end of the seat support 48 may support a seat 52, which may be made of a moldable plastic or similar material that is comfortable, durable, and easy to clean. Various seat configurations may be employed. In other examples, the seat may be contoured for comfort, such as having a saddle configuration much like a bicycle seat. Seats with added fabric or padding may be employed for added comfort (although seats with such non-rigid additions may be more difficult to clean which is significant in a hospital setting). In exemplary embodiments, the seat 52 may be rotated or swiveled on top of the seat support 48 so that the caregiver can change the facing direction, such as to turn to speak to family members, caregivers, or other persons beside the patient who may be in the hospital room. An opposite bottom end of the seat support 48 may be fitted with a traction bottom 54. The traction bottom may be configured as a rubber stopper that can provide traction control as the seat is moved from different positions, as well as prevent seat movement when a caregiver is sitting on the seat. The traction bottom further may permit the seat to glide smoothly to make adjusting the seat position easy when standing, and then sitting down holds the seat in place.

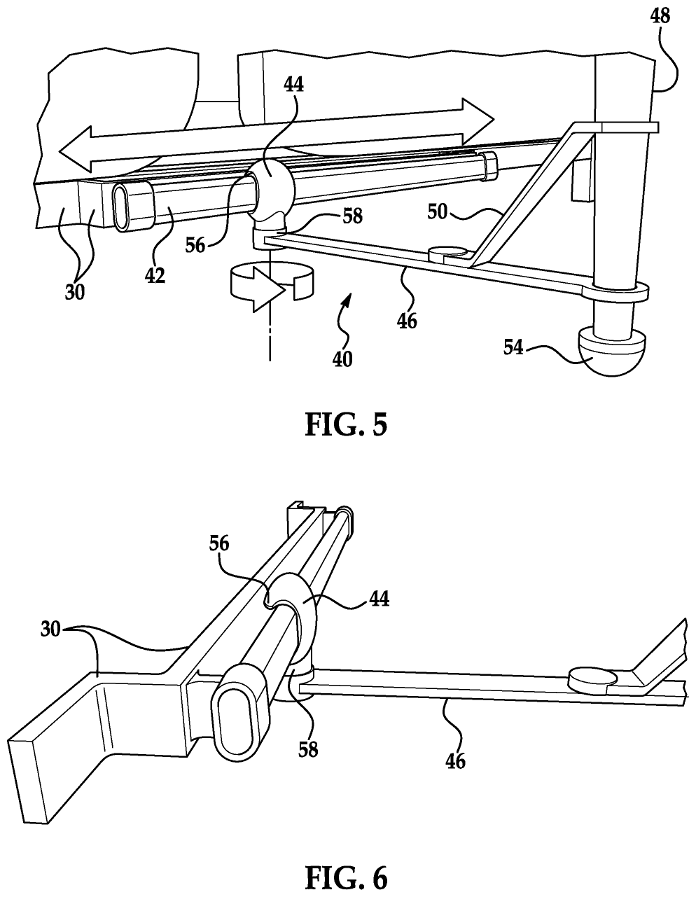

FIGS. 5 and 6 are drawings depicting more closeup views of the track and clamp portions of the seating system 40. The track 42 has sliding surfaces over which the clamp 44 can slide. Accordingly, the clamp 44 it fitted over the track 42 in a manner that permits the clamp to slide along the track. In exemplary embodiments, the clamp may be configured as a C-clamp with the open portion of the “C” shape receiving the track 42, and lips 56 extending over the track so as to provide a secured fit. A connection between the lateral bar 46 and the clamp 44 may form a rotating joint portion 58 that is formed using a pin, rod or similar connection that permits rotation of the lateral bar 46 relative to the clamp 44. In this manner, the lateral bar 46, and therefore ultimately the seat 52, can rotate or swivel relative to the clamp.

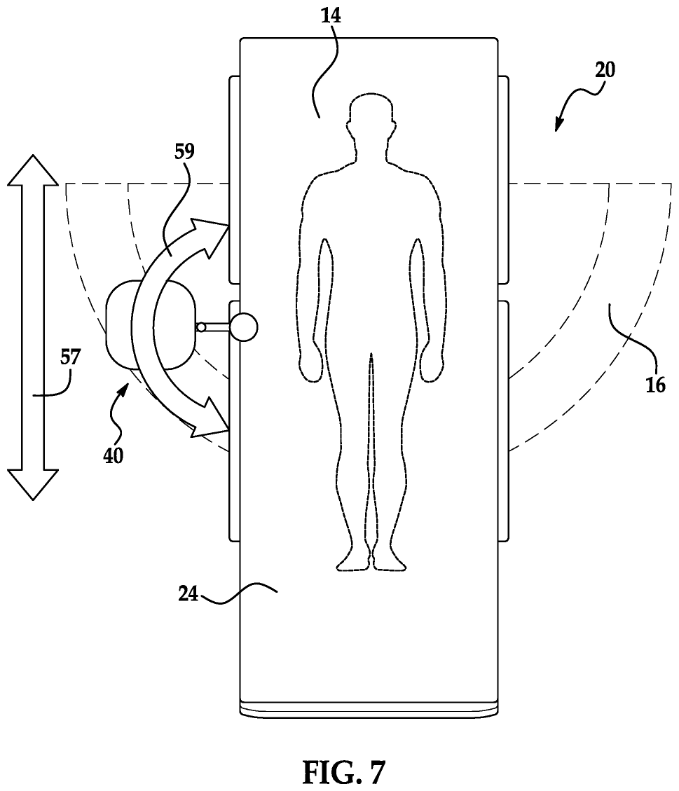

As referenced above, FIG. 2 depicts the seating system 40 in a stored state with the seat positioned with minimal intrusion into the hospital room. FIG. 2 may be compared with FIG. 7, with FIG. 7 being a drawing depicting movement of the seating system from the stored state of FIG. 2 to a seating position. FIG. 7 includes the zone of comfort 16 comparable to FIG. 1 of the 38″ to 48″ circumferential distance relative to the head portion 14 of the bed where the patient's eyes generally would be located. As seen in the various figures, and FIG. 7 in particular, the seat 52 of the seating system 40 is capable of two ranges of motion. First, the seat 52 may be moved longitudinally along the bed by sliding in a translating direction the lateral bar 46 along the track 42 (FIG. 7, arrow 57). Second, the seat 52 may be moved in a circular fashion by rotating the lateral bar 46 relative to the clamp 44 about the rotating joint clamp connection (FIG. 7, arrow 59). With these two types of motion available, as depicted in FIG. 7, the caregiver can ensure that the seating system is always positioned with the seat 52 at a seating position within the optimal zone of comfort 16. The seat position thus may be varied within the limits of motion to accommodate variations in size and precise positioning of the patient (and/or others who may be in the hospital room). In addition, when the caregiver is finished with the patient, the lateral bar 46 may be rotated back and slid or translated along the track as needed to move the seat 52 back into the stored state of FIG. 2.

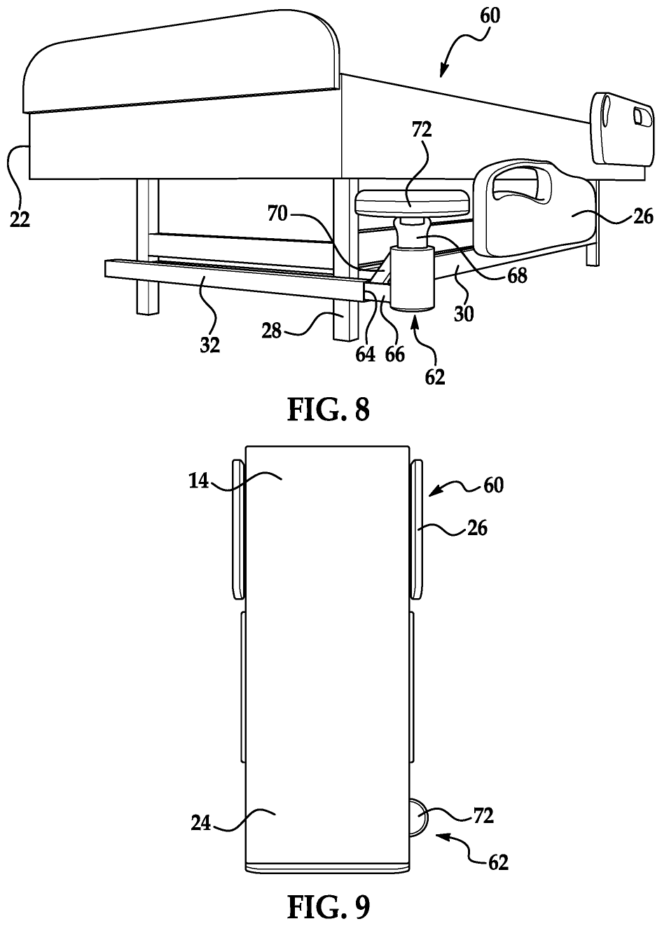

FIGS. 8-13 are drawings depicting a second exemplary hospital bed 60 in accordance with embodiments of the present invention. The basic bed components, such as the various frame and bed portion components, are largely comparable to the previous embodiment, and thus like reference numerals are used to identify like components in FIGS. 8-13 as in FIGS. 2-7. The embodiment of FIGS. 8-13 employs a different configuration of seating system. In this second example, the hospital bed 60 may include a seating system 62 that is part of the hospital bed 60. It is not uncommon in the manufacture of hospital bed frames that the metal frame components be hollowed out so as to use less material and have less weight. The embodiment of FIGS. 8-13 takes advantage of the hollow nature of one of the cross supports 32 to configure the seating system 62.

FIG. 8 depicts the seating system 62 in a stored state. In such stored state, the seating system is positioned such that a seat component of the seating system is located at least partially within the expanse of the bed frame, and particularly positioned at least partially between upper frame 22 and lower frame 28 in this example. Accordingly, in the stored state there is minimal intrusion of the seating system 62 into the hospital room. The minimal intrusion is illustrated in the plan view of FIG. 9, which shows how the seat portion of the seating system protrudes only slightly from the other bed components.

As seen in the stored state of FIG. 8, the cross support 32 may include a hollowed recess 64. A lateral bar 66 may extend through the hollowed recess from a first end to a seat support 68 at a second end of the lateral bar opposite to the first end. In the stored state of FIG. 8, only a small portion of the lateral bar is visible because the gravamen of the lateral bar is received with the hollowed recess of the cross support. The connection of the lateral bar 66 and seat support 68 may be reinforced using a reinforcement bar 70 that extends in a sloped configuration between the lateral bar and seat support. The lateral bar, seat support, and reinforcement bar may be made of any suitable rigid material, such as a rigid plastic or metal. As in the previous embodiment, aluminum components are suitable to provide adequate strength with reduced weight relative to other metal materials.

A top end of the seat support 68 may support a seat 72, which again may be made of a moldable plastic or similar material that is comfortable, durable, and easy to clean, and may be contoured and/or padded for added comfort. In exemplary embodiments, the seat 72 may be rotated or swiveled on top of the seat support 68 so that the caregiver can change the facing direction, such as to turn to speak to family members, caregivers, or other persons beside the patient who may be in the hospital room. In this embodiment, a traction bottom is not shown, although such support component may be employed in this embodiment similarly as in the previous embodiment.

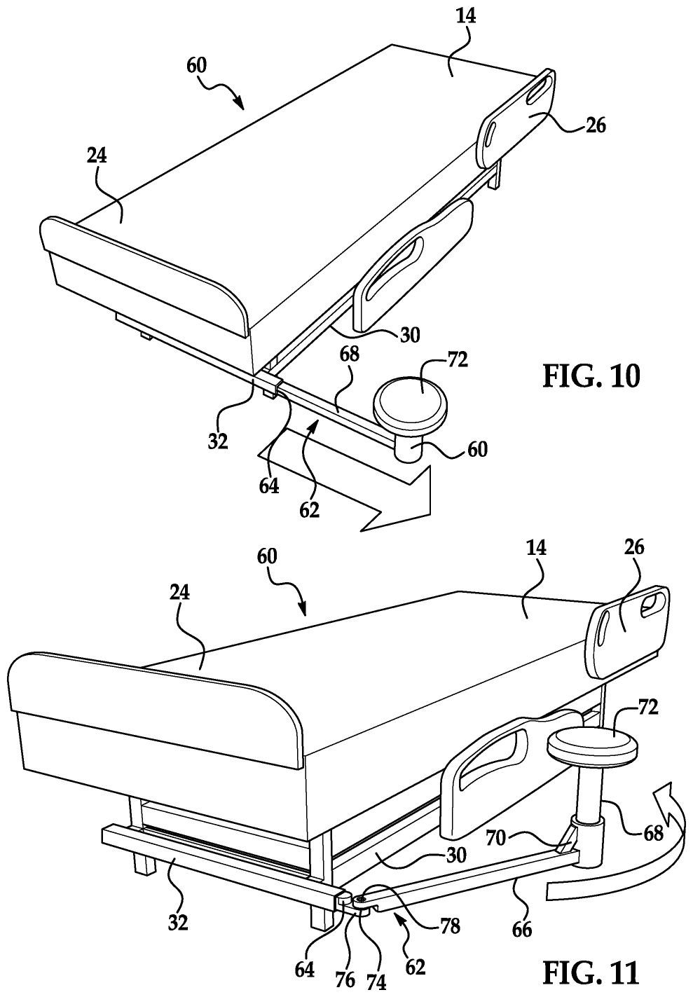

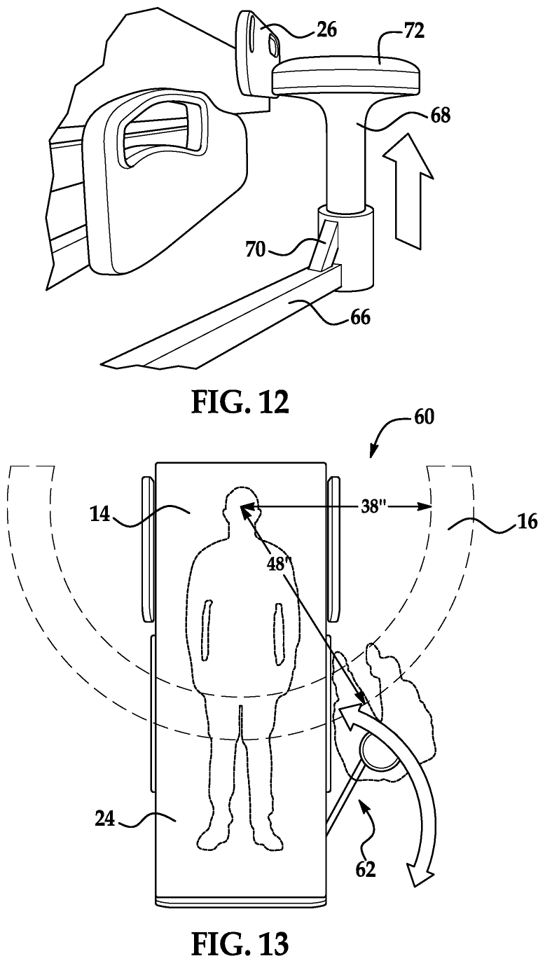

FIGS. 10-12 are drawings depicting operation of the second embodiment employing the seating system 62. As seen in FIG. 10, in a first step of operation the lateral bar 66 may be pulled through the hollow cross support to extend outward from such cross support. The lateral bar is pulled outward in such manner to maximum extension to expose a rotating joint portion 74 located at the first end of the lateral bar, which is shown in FIG. 11. The rotating joint portion 74 includes a joint base 76 that is fixed to the lateral bar 66 using a pin 78 or similar rod-like connection that permits rotation of the lateral bar. In this manner, as shown in FIG. 11 the lateral bar 66, and therefore ultimately the seat 72, can rotate or swivel relative to the rotating joint portion 74. In the stored state of FIG. 8, the seat 72 may be in a lowered position so as to fit within the bed frame components. As seen in FIG. 12, the seat 72 may be raised by raising the seat in a telescoping manner from the seat support 68. By having a seat that can be raised or lowered, the seat can be positioned to fit within the bed frame components for space-saving storage in the stored state, but can be raised to be more at patient eyelevel in the seating position during use by the caregiver.

As referenced above, FIG. 8 depicts the seating system 62 in a stored state with the seat positioned with minimal intrusion into the hospital room. FIG. 8 may be compared with FIG. 13, with FIG. 13 being a drawing depicting movement of the seating system from the stored state of FIG. 8 to a seating position. FIG. 13 again includes the zone of comfort 16 comparable to FIG. 1 of the 38″ to 48″ circumferential distance relative to the head portion 14 of the bed where the patient's eyes generally would be located. As seen in the various figures, and FIG. 13 in particular, the seat 72 may be swivel via rotation of the lateral bar 66 relative to the rotating joint portion 74, which permits the caregiver to position the seating system with the seat 72 at a seating position within the optimal zone of comfort 16. In addition, when the caregiver is finished with the patient, the lateral bar 66 may be rotated as needed to permit sliding the lateral bar back through the hollowed space to the cross support 32, thereby returning the seating system to the stored state of FIG. 8.

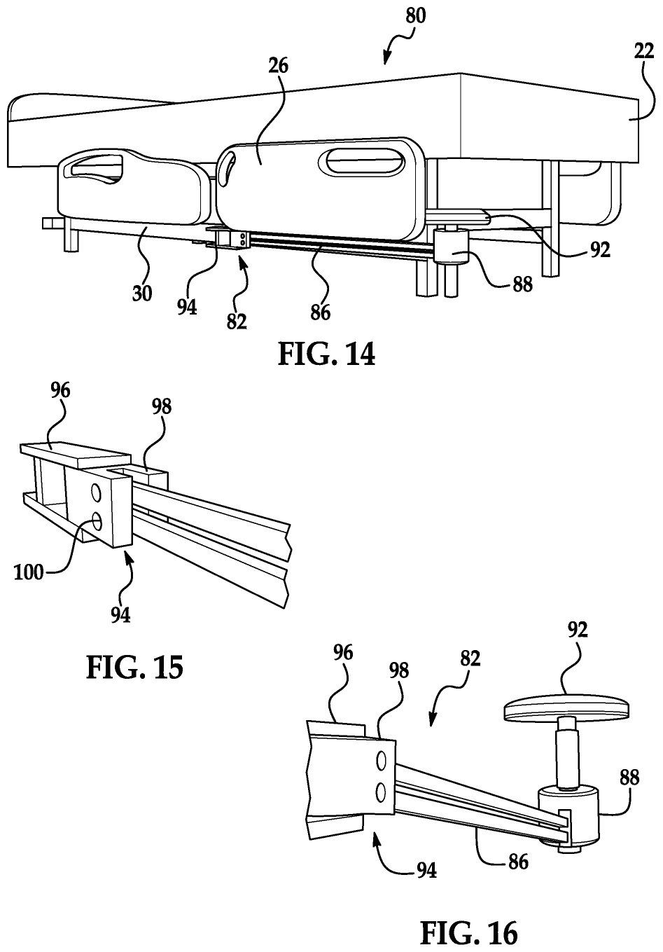

The embodiments of FIGS. 2-7 and 8-13 may be characterized as having a single pivot seating system, whereby a single pivot point where the lateral bar rotates at a rotating joint portion provides for movement of the seat (the first embodiment adds sliding translation along the track). FIGS. 14-20 are drawings depicting a third exemplary hospital bed 80 in accordance with embodiments of the present invention, which constitutes an alternative single pivot seating system. The basic bed components again are largely comparable to previous embodiments, and thus like reference numerals are used to identify like components in FIGS. 14-20. The embodiment of FIGS. 14-20 employs yet another different configuration of seating system. In this third example, the hospital bed 80 may include a seating system 82 that is part of the hospital bed 80.

FIG. 14 depicts the seating system 82 in a stored state. In such stored state, the seating system is positioned at least partially within the bed frame, and particularly positioned such that a seat component of the seating system is located at least partially within the expanse of the bed frame, and particularly positioned at least partially between upper frame 22 and lower frame 28 in this example. Accordingly, in the stored state there is minimal intrusion of the seating system 82 into the hospital room similarly as in previous embodiments. As further detailed below, the third embodiment also has a single pivot configuration, although the pivot location and structure differs.

As seen in the stored state of FIG. 14, the single pivot point is provided by connection to one of the longitudinal supports 30. A lateral bar structure 86 may extend from a joint structure 94 at a first end fixed to the longitudinal support to a seat support 88 at a second end of the lateral bar opposite from the first end. In this particular example, the lateral bar structure 86 is actually a double bar for reinforcement. The lateral bar structure and seat support may be made of any suitable rigid material, such as a rigid plastic or metal. As in the previous embodiments, aluminum components are suitable to provide adequate strength with reduced weight relative to other metal materials.

A top end of the seat support 88 may support a seat 92, which again may be made of a moldable plastic or similar material that is comfortable, durable, and easy to clean, and may be contoured and/or padded for added comfort. In exemplary embodiments, the seat 92 may be rotated or swiveled on top of the seat support 88 so that the caregiver can change the facing direction, such as to turn to speak to family members, other caregivers or other persons beside the patient who may be in the hospital room. In this embodiment, a traction bottom 90 is provided similarly as in the first embodiment to provide traction of the seating system relative to the floor.

FIGS. 15 and 16 depict close-up views of a joint structure 94 that is employed in this particular embodiment. The joint structure 94 may include a first joint 96 and a second joint 98. It will be appreciated that the joint structure 94 may be employed in other embodiments of seating systems to the extent compatible. The first joint 96 may be configured as a clamp with a vertical pin or similar rod structure (not visible in this figure) to which the second joint 98 is connected in a rotatable fashion. The second joint 98 may be configured as another clamp having one or more horizontal pins or similar rod structures 100 to which the lateral bar structure 86 may be fixed at the first end of the lateral bar. The first joint 96, with the vertical pin or rod, permits rotational circumferential movement of the lateral bar structure 86 relative to the longitudinal support 30 of the bed frame, and therefore the seat 92, relative to the first joint in substantially a horizontal plane. The second joint 98 with the horizontal pins, as seen particularly in FIG. 16, permits a vertical movement of the lateral bar structure 86, and therefore the seat 92, between a lower position on the floor for seating and an upper off-the-floor position for the stored state. A spring device may bias the lateral bar structure in the up position for storage, with the caregiver pushing or by weight lowering the seat to the floor to provide better support and stability when seating.

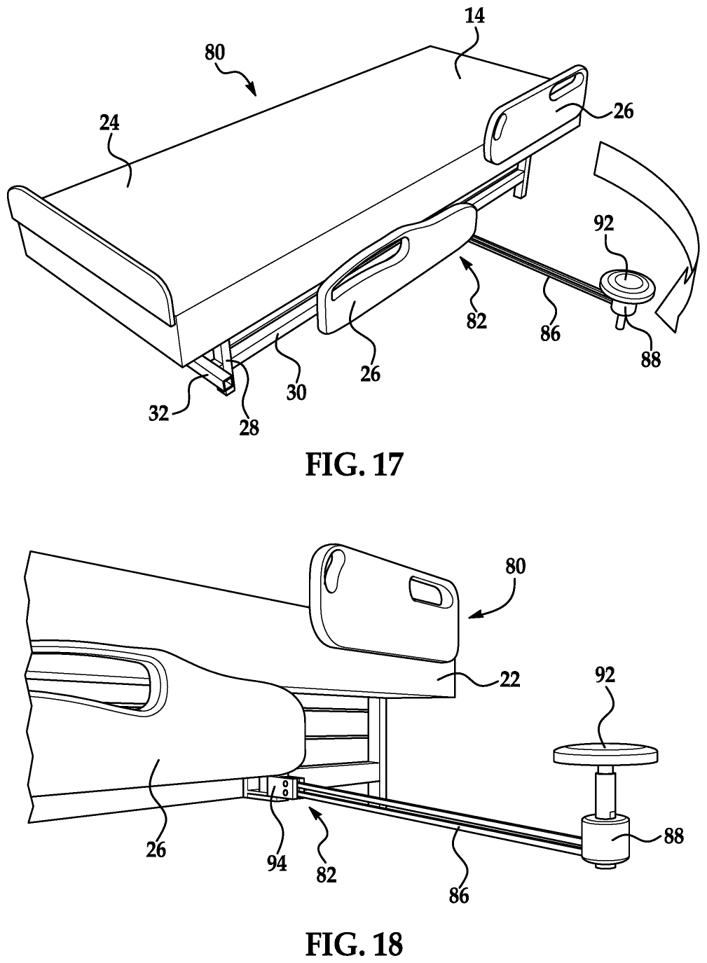

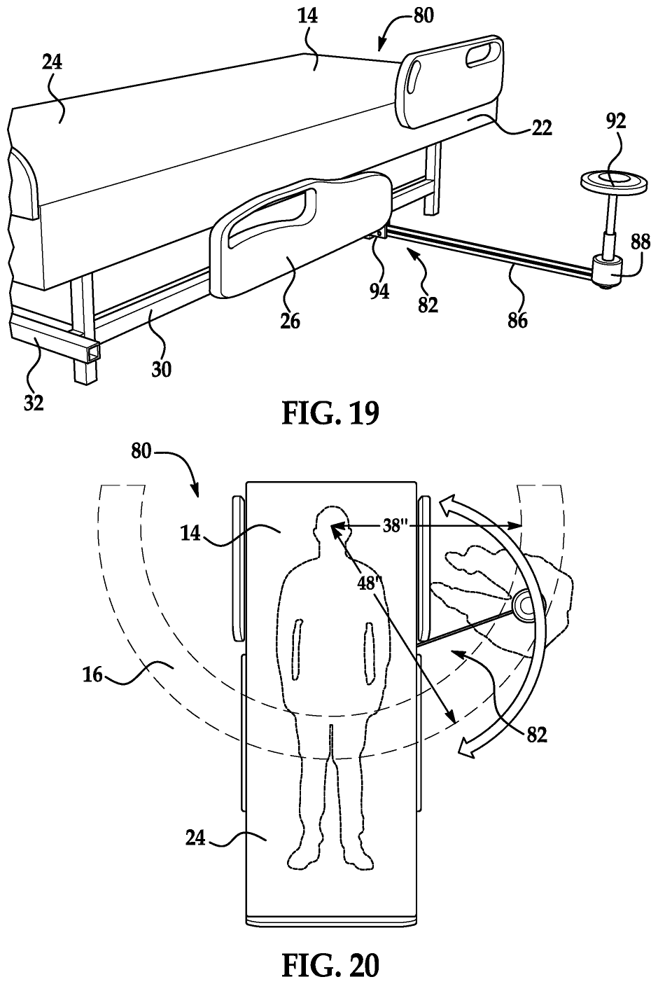

FIGS. 17-19 are drawings depicting operation of the third embodiment employing the seating system 82. As seen in FIG. 17, in a first step of operation the lateral bar structure 86 may be rotated via the first joint 96 out of the stored state, as illustrated by the arrow in the figure. As seen in FIG. 18, in a second step of operation the lateral bar structure may be pressed downward via operation of the second joint 98 to engage the seating system against the floor for traction at the desired position. As seen in FIG. 19, in a third step of operation the seat 92 then may be raise by raising the seat in a telescoping manner from the seat support 88. By having a seat that can be raised or lowered, as in previous embodiments the seat can be positioned to fit within the bed frame components for space-saving storage, but can be raised to be more at patient eyelevel during use by the caregiver.

As referenced above, FIG. 14 depicts the seating system 82 in a stored state with the seat positioned with minimal intrusion into the hospital room. FIG. 14 may be compared with FIG. 20, with FIG. 20 being a drawing depicting movement of the seating system from the stored state of FIG. 14 to a seating position. FIG. 20 again includes the zone of comfort 16 comparable to FIG. 1 of the 38″ to 48″ circumferential distance relative to the head portion 14 of the bed where the patient's eyes generally would be located. As seen in the various figures, and FIG. 20 in particular, the seat 92 may be swiveled via rotation of the lateral bar structure 86 relative to the first joint 96, which permits the caregiver to position the seating system with the seat 92 at a seating position within the optimal zone of comfort 16. In addition, when the caregiver is finished with the patient, the lateral bar structure 86 may be rotated as needed to return the seating system to the stored state of FIG. 14. FIG. 20 also may be compared to FIG. 13 above for the previous single-pivot embodiment. By positioning the pivot point along the longitudinal support, the embodiment of FIG. 20 permits a wider range of movement within the zone of comfort 16, as compared to pivoting the seating system from the cross support as seen in FIG. 13.

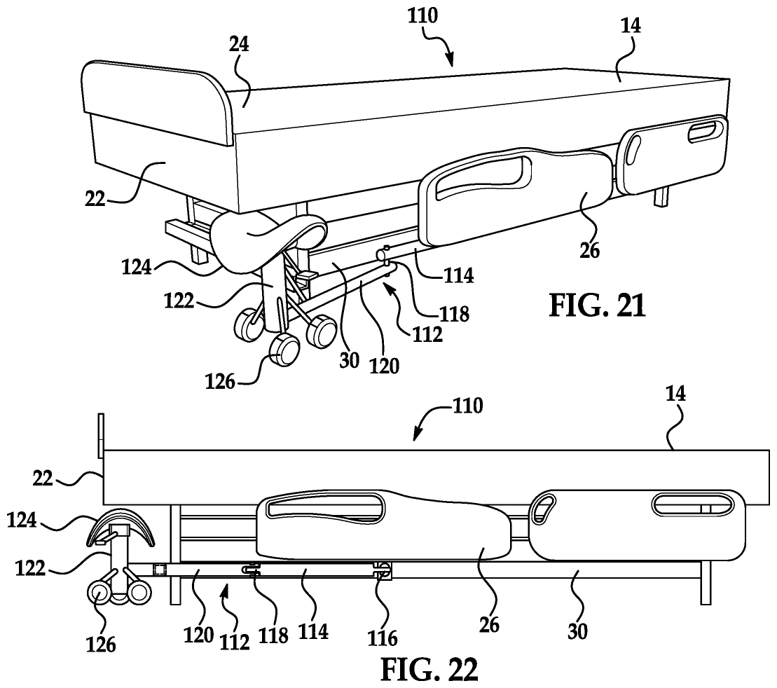

As referenced above, the previous embodiments may be characterized as single-pivot embodiments in that the lateral bar structure with the seat can rotate about a single pivot point. The first embodiment adds translational movement of sliding along the track. In another exemplary embodiment, a double-pivot structure may be employed that permits rotation about two pivot points. FIGS. 21-28 are drawings depicting a fourth exemplary hospital bed 110 in accordance with embodiments of the present invention, which constitutes a double pivot seating system. The basic bed components again are largely comparable to previous embodiments, and thus like reference numeral are used to identify like bed components in FIGS. 21-28. The embodiment of FIGS. 21-28 employs yet another different configuration of seating system. In this second example, the hospital bed 110 may include a seating system 112 that is part of the hospital bed 110.

FIG. 21 depicts the seating system 112 in a stored state. In such stored state, the seating system is positioned at least partially within the bed frame, and particularly positioned such that a seat component of the seating system is located at least partially within the expanse of the bed frame, and particularly positioned at least partially between upper frame 22 and lower frame 28 in this example. Accordingly, in the stored state there is minimal intrusion of the seating system 112 into the hospital room similarly as in previous embodiments. As further detailed below, the fourth embodiment has a double pivot configuration, which permits rotation about two pivot points to configure the seat. Generally, to provide a double pivot system, the lateral bar may be divided into segments, with a segment of the lateral bar being connected to the seat support that supports the seat component. Multiple rotating joint components may connect adjacent segments of the lateral bar, and may connect a first segment of the lateral bar to the longitudinal support, thereby forming a double pivot mechanism. The seating system is moveable between the stored state and the seating position by rotating the lateral bar relative to the longitudinal support and rotating segments of the lateral bar relative to each other via the rotating joints.

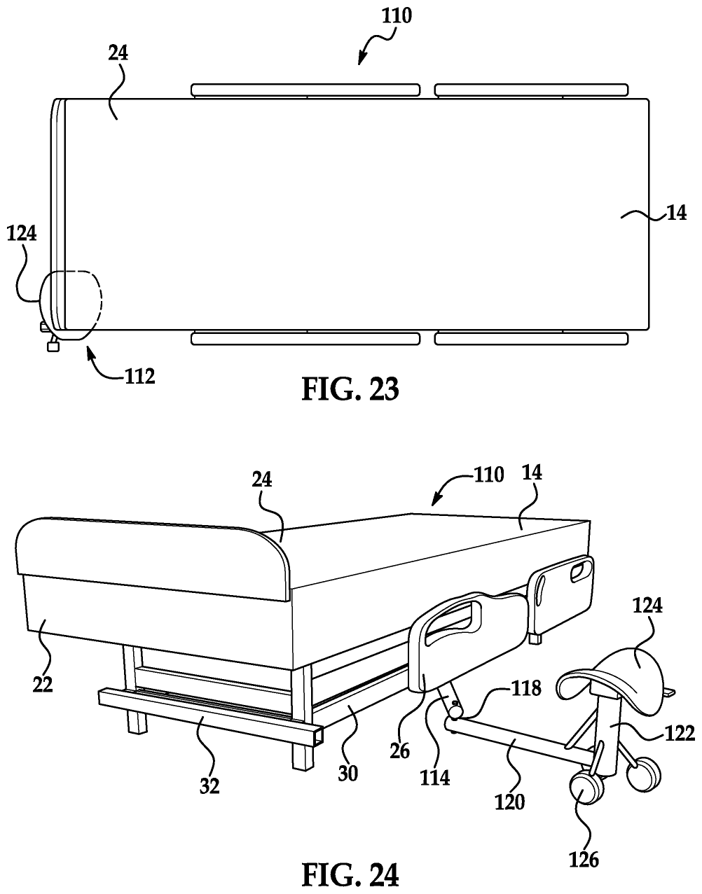

FIG. 22 is a drawing depicting a side view that illustrates the double pivot configuration of this embodiment, and FIG. 23 is a drawing depicting a corresponding top view. Referring to FIGS. 21-23, the double pivot mechanism is provided by forming a lateral bar structure in multiple components. A first pivot point is provided by connection of a first lateral bar component 114 at a first end to one of the longitudinal supports 30. The first lateral bar component 114 may extend from a rotating first joint 116 at a first end that is fixed to the longitudinal support. The first joint 116, similar to joints in previous embodiments, may employ a pin or rod structure that permits swiveling or rotation of the first lateral bar component 114 relative to the longitudinal support 30 via the first rotating joint. The first lateral bar component 114 may extend from the first joint 116 to a rotating second joint 118 at a second end. A second lateral bar component 120 may then extend from the second joint 118 at a first end to a seat support 122 at a second end. Like the first joint 116, the rotating second joint 118, similar to joints in previous embodiments, may employ a pin or rod structure that permits swiveling or rotation of the second lateral bar component 120 relative to the first lateral bar component 114. In this manner, a double pivot structure is provided by rotations of both lateral bar components relative to the respective rotating joints. The lateral bar components, joints, and seat support again may be made of any suitable rigid material, such as a rigid plastic or metal. As in the previous embodiments, aluminum components are suitable to provide adequate strength with reduced weight relative to other metal materials.

A top end of the seat support 122 may support a seat 124, which again may be made of a moldable plastic or similar material that is comfortable, durable, and easy to clean and may be contoured and/or padded for added comfort. In this embodiment, the seal has the saddle configuration referenced above for added support and comfort. In exemplary embodiments, the seat 124 may be rotated or swiveled on top of the seat support 122 so that the caregiver can change the facing direction, such as to turn to speak to family members, other caregivers, or other persons beside the patient who may be in the hospital room. In this embodiment, a second end of the seat support 122 is provided with wheels 126 as an alternative to a stopper-like traction bottom used in connection with previous embodiments. It will be appreciated that wheels and traction bottoms may be interchanged in the different seating system embodiments as may be desirable to a particular user.

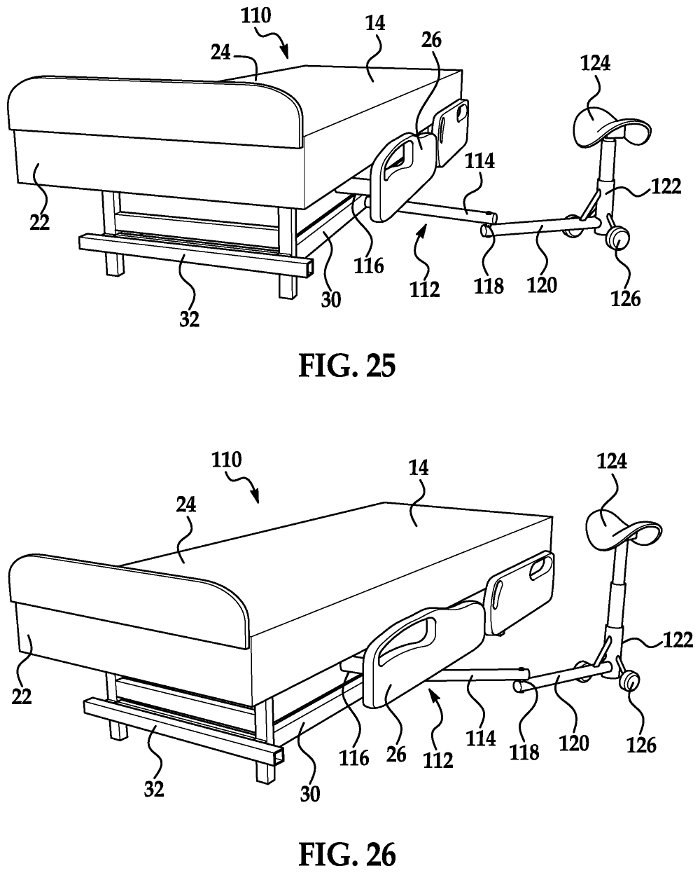

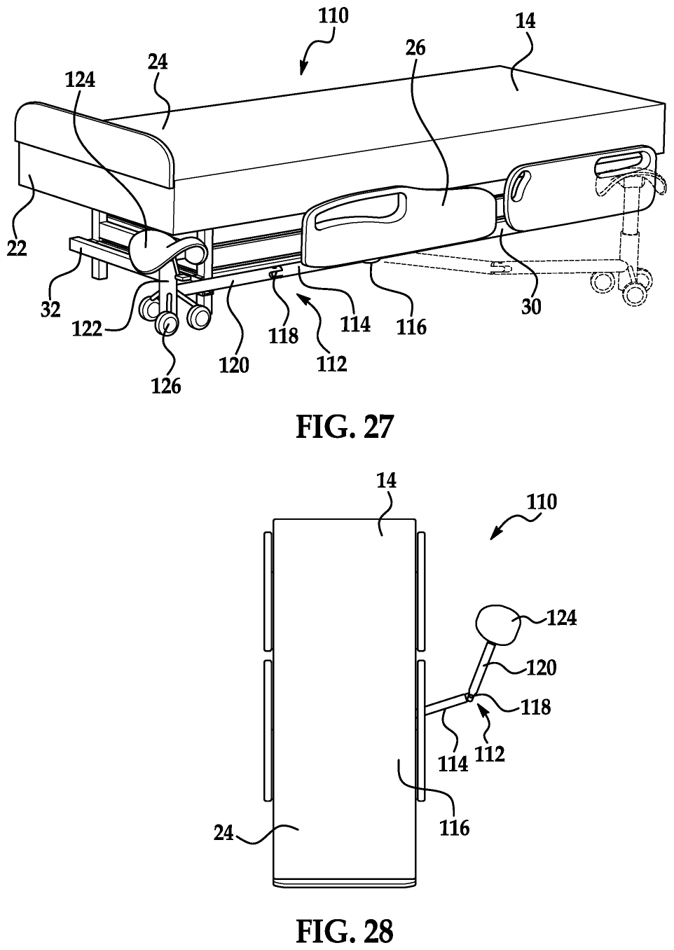

FIGS. 24-26 are drawings depicting operation of the fourth embodiment employing the seating system 112. As seen in FIG. 24, in a first step of operation one or both the lateral bar components 114 and 120 may be rotated via the respective rotating first and second joints 116 and 118 out of the stored state. As seen in FIG. 25, in a second step of operation the lateral bar components may be rotated further until the seat is positioned at or approaching a desired location within the zone of comfort. As seen in FIG. 26, as a third step of operation additional rotation of the lateral bar components about the joints can fine tune or additionally adjust the position of the system to any optimal position. In addition, the seat 124 may be raised by raising the seat in a telescoping manner from the seat support 122. By having a seat that can be raised or lowered, as in previous embodiments the seat can be positioned to fit within the bed frame components for space-saving storage in the stored state, but can be raised to be more at patient eyelevel to the seating position during use by the caregiver. The double pivot nature of the present embodiment provides an enhanced range of positioning of the seating system. FIGS. 27 and 28 are drawings depicting such range of positioning from a perspective view (FIG. 27) and a plan view (FIG. 28). With two lateral bar components rotating about two respective rotating joints, a wider range of optimal positioning within the zone of comfort can be achieved.

Comparing the various embodiments, the double pivot nature of the fourth embodiment can be beneficial in affording a greater range of positioning, but can be more complex to manufacture and assemble as compared to the single pivot embodiments. The first embodiment can perhaps represent a balance between range of motion versus complexity, by adding to the single pivot motion a second translational motion by sliding movement along the track. Such a dual motion perhaps affords less motion range that the full double pivot embodiment, but is easier to manufacture and assemble and provides enhanced motion relative to the more limited single pivot embodiments. The second embodiment perhaps gives the least range of motion, but takes advantage of the existing structure of the typical hollowed cross support without having to add the additional track or other structures. The third embodiment also is more simple with the joint structure being attached to the longitudinal support with a single pivot motion. Accordingly, different embodiments may be more or less advantageous depending upon particular circumstances of the restrictions and freedoms that any given hospital room and/or base bed structure may have.

Interactions between caregivers and patients can vary in duration, and sometimes can be very brief. For such brief interactions, it may not be convenient or efficient to move the above seating systems out from the stored state to the seating position. However, even during brief interactions, it still may be desirable for the caregiver to be seated at approximately eyelevel with the patient, and within the referenced zone of comfort. Accordingly, aspects of the present invention include a hospital bed employing a flip seat configuration that is incorporated into the bed components, that can be more easily moved from a stored state to a seating position for briefer caregiver-patient interactions.

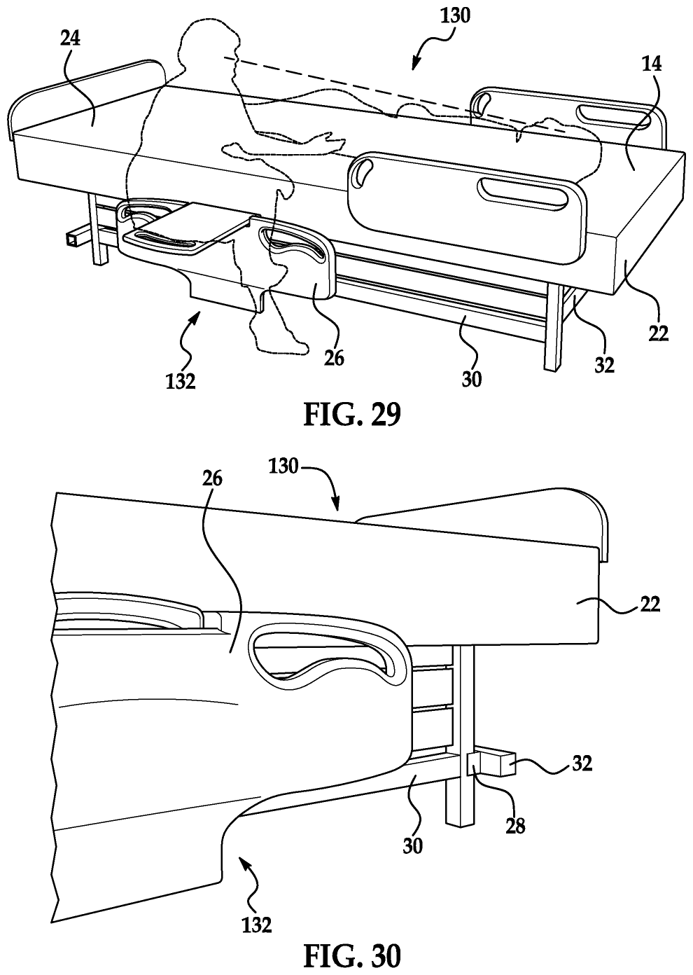

FIGS. 29-36 are drawings depicting a fifth exemplary hospital bed 130 in accordance with embodiments of the present invention, which includes a flip seat configuration with a flip seat that is incorporated into at least one of the bedrails 26. The basic bed components again are largely comparable to previous embodiments, and thus like reference numerals are used to identify like components in FIGS. 29-36. The embodiment of FIGS. 29-36 employs yet another different configuration of seating system. In this fifth example, the hospital bed 130 may include a seating system 132 that is part of the hospital bed 130, and more particularly is incorporated into a bedrail 26.

FIG. 29 is a drawing depicting the exemplary hospital bed 130 with the seating system 132 in a seating position, with caregiver and patient shadows included. FIG. 30 is a drawing depicting a related portion of the hospital bed 130 with the seating system 132 in the stored state. In such stored state, the seating system is positioned at least partially within the bed frame, and particularly positioned substantially within the bedrail 26 in this example. Accordingly, in the stored state there is minimal intrusion of the seating system 132 into the hospital room similarly as in previous embodiments.

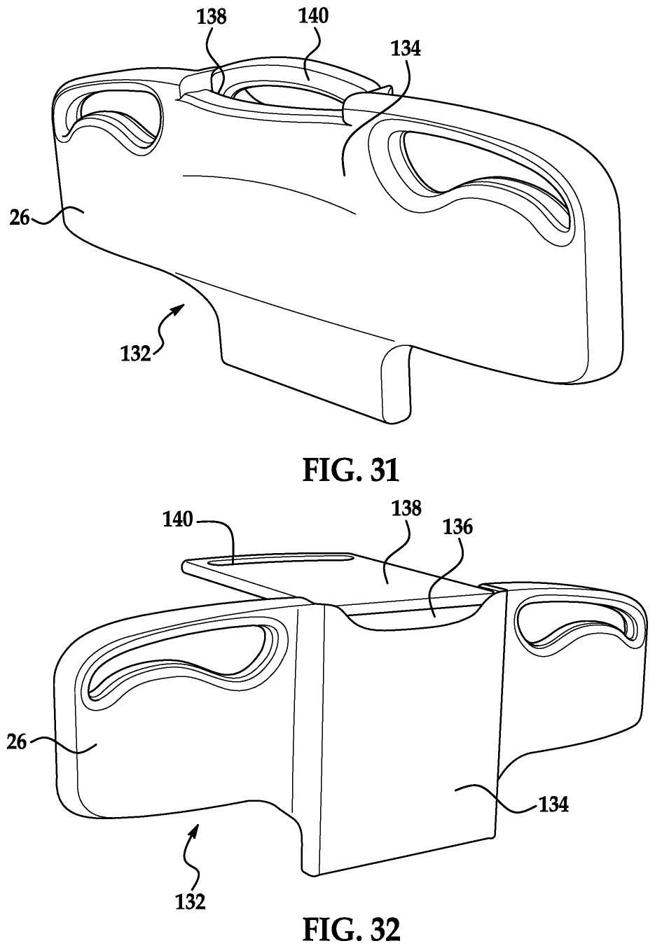

FIGS. 31 and 32 are drawings depicting the seating system 132 isolated from the other bed components for ease of illustration, with FIG. 31 showing the seating system in the stored state and FIG. 32 showing the seating system in the seating position. As seen in the figures, the seating system 132 may include the bedrail 26, which in this example has an integrated seat housing 134. The seat housing 134 defines a spacing 136 (see particularly FIG. 32) that is configured to enclose a seat portion 138 in the stored state. In this manner, as shown in FIG. 31, in the stored state the seat portion 138 is substantially contained within the seat housing 134 of the bedrail 26 so as not to protrude into the broader hospital room.

The seat portion 138 may include a gripping portion 140, such as a handle, that extends from the seat housing 134 when the seat portion 138 is in the stored state. With such configuration, the gripping portion or handle remains accessible to a user in the stored stated with minimal extension from the bedrail (see FIG. 31). The seat portion 138 may be moved from the stored state to the seating position by pulling the handle to slide the seat portion 138 from the seat housing 134, and flipping the seat portion downward to the seating position.

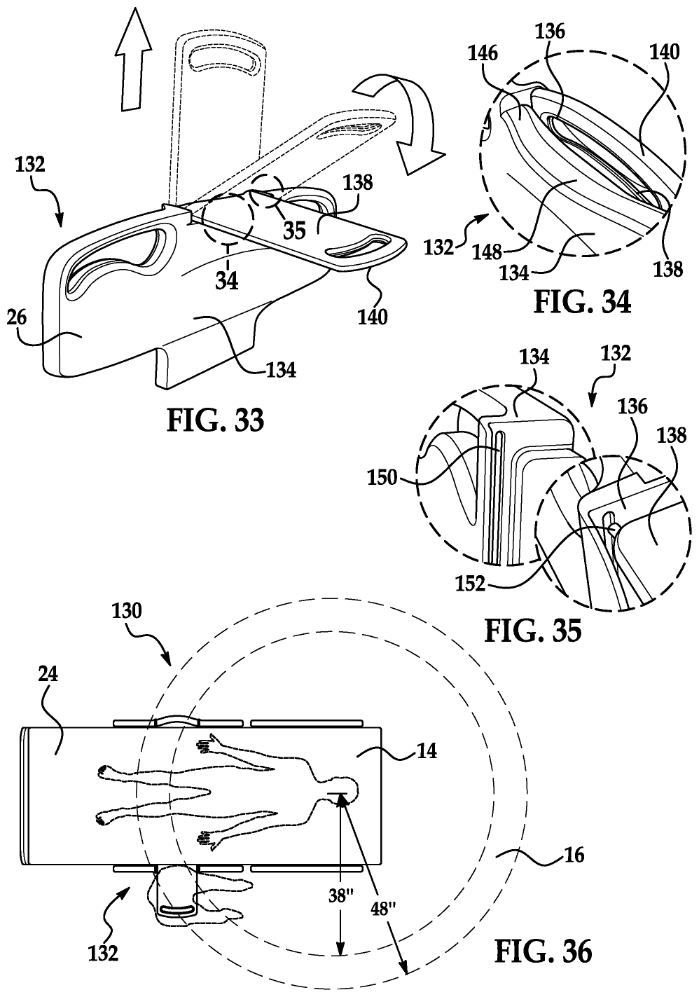

FIG. 33 is a drawing depicting operation of the fifth embodiment employing the seating system 132 in more detail. In a first step of operation, the seat portion 138 is pulled upward from the seat housing 134 using the handle 140, as shown by the arrow 142. In a second step of operation, the seat portion 138 is flipped downward to the seating position as shown by the second arrow 144. The operations may be reversed to return the seat portion 138 to the stored state located within the seat housing 134.

FIG. 34 is a drawing depicting a closeup view of a portion of the seating system 132 with the seat portion 138 in the stored state. An upper end of the seat housing 134 constitutes a seat ledge 146 that can support the seat portion 134 with a sitting user when the seat portion is in the seating position. Typically, the seat portion 138 and the bedrail 26 (including the seat housing 134) may be made of common materials, and moldable rigid plastics are suitable materials because they are strong enough to support the caregiver and easy to clean and manipulate. The seat ledge 146 may include a cutaway or concave portion 148 that permits access to the handle 140 when the seat portion 138 is in the stored state, as referenced above.

FIG. 35 is a drawing depicting another closeup view of a portion of the seating system 132 that illustrates the mechanical connections and interface of the seat housing and seat portion. The seat housing 134 may define a seat track 150, and the seat portion 138 may include an extension pin 152. The extension pin 142 may be received within the seat track 150, such that pin slides within the track when the seat portion is moved out from and into the seat housing. In addition, the pin-track interaction permits the extension pin to rotate within the seat track to permit the flipping movements of the seat portion into and from the seating position as described above.

As referenced above, FIG. 30 depicts the seating system 132 in a stored state with the seat positioned with minimal intrusion into the hospital room. FIG. 30 may be compared with FIG. 36, with FIG. 36 being a drawing depicting the seating system reconfigured from the stored state of FIG. 30 to a seating position. FIG. 36 again includes the zone of comfort 16 comparable to FIG. 1 of the 38″ to 48″ circumferential distance relative to the head portion 14 of the bed where the patient's eyes generally would be located. As seen in FIG. 36, when in the seating position the seat portion 138 is located within the zone of comfort 16. The flip seat configuration is fixed at such position in contrast to the previous pivoting embodiments, and thus the flip seat configuration is less flexible in positioning. The flip seat configuration, however, may be more suitable for brief interactions as the flip seat is easier to manipulate as compared to the previous pivoting seating system embodiments.

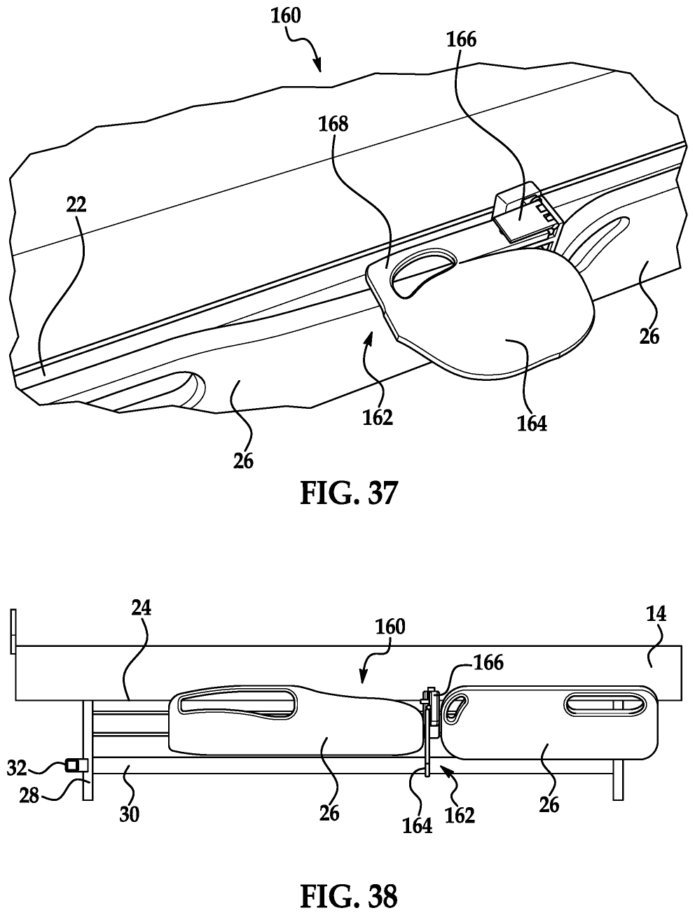

FIGS. 37-42 are drawings depicting a sixth exemplary hospital bed 160 in accordance with embodiments of the present invention, which includes an alternative flip seat configuration. The basic bed components again are largely comparable to previous embodiments, and thus like reference numerals are used to identify like components in FIGS. 37-42. The embodiment of FIGS. 37-42 employs yet another different configuration of seating system. In this sixth example, the hospital bed 160 may include a seating system 162 that is part of the hospital bed 160, and more particularly is incorporated into the upper frame 22 adjacent to or between one or more bedrails 26.

FIG. 37 is a drawing depicting the exemplary hospital bed 130 with the seating system 132 in a seating position. As further detailed below, a seating portion of the seating system may be flipped and moved into the seating position from the stored state, and vice versa. FIG. 38 depicts the hospital bed 160 with the seating system 162 in the stored state. In such stored state, the seating system is positioned at least partially within the bed frame, and particularly positioned adjacent the upper frame 22 adjacent to or between one or more bedrails, such as for example opposing bedrails 26 in this example. Accordingly, in the stored state there is minimal intrusion of the seating system 162 into the hospital room similarly as in previous embodiments.

As seen in FIGS. 37-38, the seating system 162 may include a seat portion 164 that is fixed to a pivotable clamp 166. The seat portion 164 may be rotated about the pivotable clamp 166 to flip up the seat portion from the seating position in FIG. 37, and then the clamp with the attached seat portion may be translated downward to move the seat portion to the stored state position of FIG. 38 adjacent to or between the opposing bedrails 26. In this manner, as shown in FIG. 38, in the stored state the seat portion 164 is substantially contained adjacent to or between the opposing bedrails 26 so as not to protrude into the broader hospital room.

The seat portion 164 may include a gripping portion 168, such as a handle, that extends from the seat portion 164 when the seat portion is in the stored state. With such configuration, the gripping portion or handle remains accessible to a user in the stored stated with minimal extension from the frame components or bedrails (see FIG. 38). The seat portion 164 may be moved from the stored state to the seating position by pulling the handle to slide the seat portion 164 from the stored state position, and flipping the seat portion downward to the seating position.

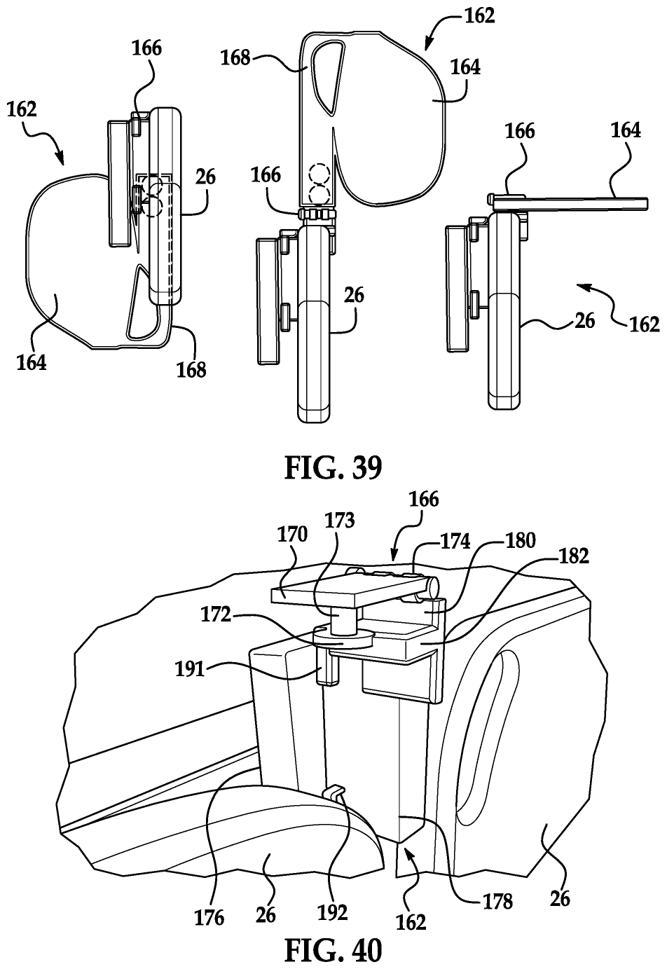

FIG. 39 is a drawing depicting operation of the sixth embodiment employing the seating system 162 in more detail. In a first step of operation, the seat portion 164 is pulled upward from between the bedrails using the handle 168, as shown by the progression of the first view to the second view of FIG. 39. In a second step of operation, the seat portion 164 is flipped downward to the seating position as shown in the third view of FIG. 39. The operations may be reversed to return the seat portion 164 to the stored state located adjacent the upper frame 28 between the bedrails 26.

FIG. 40 is a drawing depicting a closeup view of a portion of the seating system 162 including the pivotable clamp 166, which demonstrates the mechanical connections and interface components of the seating system. In the view of FIG. 40, the seat portion 164 is removed for ease of illustration. The clamp 166 may include a first clamp plate 170 and an opposing second clamp plate 172. A connecting rod 173 extends between the clamp plates, which as further shown below, provides a mounting interface for connection of the seating portion. The clamp 166 further may include a rotating hinge 174 that permits the clamp plates, and the connected seat portion, to rotate outward to permit the flipping movement described above with respect to FIGS. 37-39.

The seating system 162 further may include a mounting base 176, which mounts the seating system 162 to the upper frame 22. The mounting base 176 may include a slide bar 178 upon which the pivotable clamp 166 is mounted. The clamp 166 further may include a sliding support 180 that is slidably fixed to the slide bar 178. In operation, the slide support 180 is moveable in a vertical direction to slide along the slide bar to move the connected seating portion into and out from the stored state. The pivotable clamp further may include a lateral support plate 182, which cooperates with the clamp plates 170 and 172 to support the seating portion when the seating portion is in the seating position.

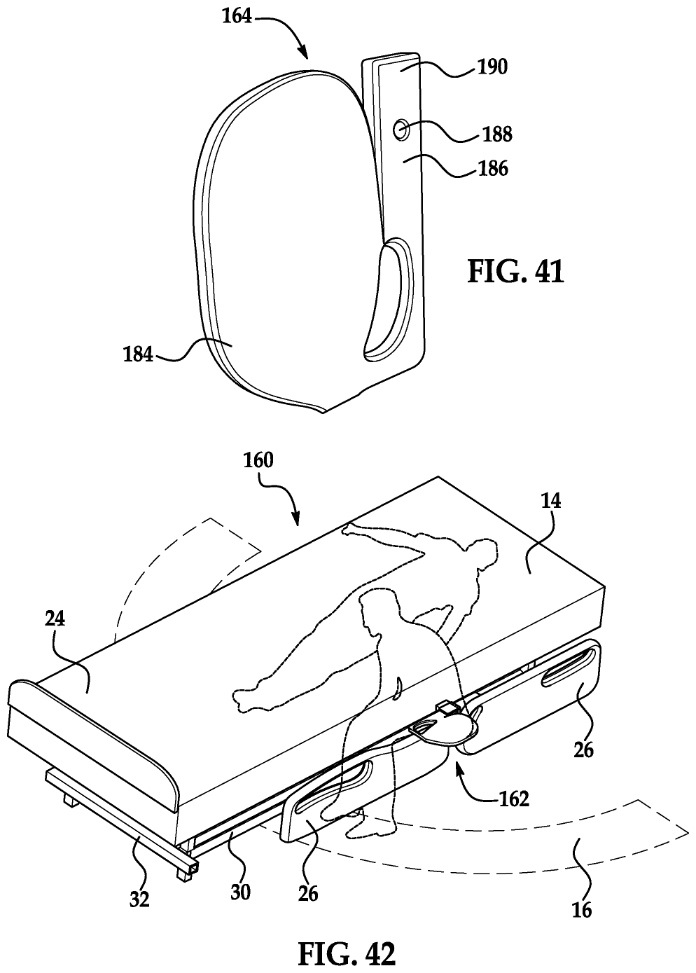

FIG. 41 is a drawing depicting a perspective view of the seat portion 164 in isolation. The seat portion 164 may include a seat 184 connected to a mounting bar 186. The mounting bar 186 may include a mounting hole 188 that is configured to receive the connecting rod 173. The seating portion may be removed from the clamp 166 for cleaning or replacement as needed. When the seat portion 164 is mounted to the clamp 166, the mounting bar is mounted to the clamp with the connecting rod 173 extending through the mounting hole 188. In this manner, the mounting bar is fixed between the clamp plates 170 and 172 to support the seat portion, with an end portion 190 of the mounting bar resting against the lateral support plate 182 to provide additional support particularly when the seat portion is in the seating position.

When in the stored state, the slide support is positioned at its lowermost level relative to the slide bar, with the handle or gripping portion 168 extending upward and accessible to the user. A pole 191 on the mounting plate may interact against a stopper 192 on the slide bar (see FIG. 40) to set the position of the seating system in the stored state. To move the seat portion 164 from the stored state to the seating position, a user may pull the handle upward, by which the clamp 166 (and thus the connected seat portion 164) slides upward via the sliding support 180 sliding upward along the slide bar 178. Once the clamp 166 has cleared the bedrails 26, the seat portion 164 may be flipped to the seating position as shown in FIGS. 37 and 39 (third view), by rotating the clamp plates (and thus again the connected seat portion) via the rotating hinge 174.

As referenced above, FIG. 38 depicts the seating system 162 in a stored state with the seat positioned with minimal intrusion into the hospital room. FIG. 38 may be compared with FIG. 42, with FIG. 42 being a drawing depicting the seating system reconfigured from the stored state of FIG. 38 to a seating position, and showing shadow depictions of a caregiver and patient. FIG. 42 again includes the zone of comfort 16 comparable to FIG. 1 of the 38″ to 48″ circumferential distance relative to the head portion 14 of the bed where the patient's eyes generally would be located. As seen in FIG. 42, when in the seating position the seat portion 164 is located within the zone of comfort 16. Similarly to the fifth embodiment, the flip seat configuration of the sixth embodiment is fixed at such position in contrast to the pivoting seat embodiments, and thus this flip seat configuration also is less flexible in positioning. The flip seat configuration of the sixth embodiment, however, also may be more suitable for brief interactions as the flip seat is easier to manipulate as compared to the pivoting seating system embodiments.

It will be appreciated that the various embodiments of pivoting seating system configurations and flip seat configurations may be combined. For example, a given hospital bed configuration may incorporate multiple pivoting seating systems, such as on different sides of the bed and associated with the different longitudinal and cross supports that constitute the frame components. In addition, a given hospital bed configuration further may include one or more of the flip seat seating systems in combination with one or more of the pivoting seating systems. All such varied combinations may be utilized within the principles and scope of this disclosure.

Although the invention has been shown and described with respect to a certain embodiment or embodiments, equivalent alterations and modifications may occur to others skilled in the art upon the reading and understanding of this specification and the annexed drawings. In particular regard to the various functions performed by the above described elements (components, assemblies, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein exemplary embodiment or embodiments of the invention. In addition, while a particular feature of the invention may have been described above with respect to only one or more of several embodiments, such feature may be combined with one or more other features of the other embodiments, as may be desired and advantageous for any given or particular application.

1. A bed comprising:
a bed portion;
a frame that supports the bed portion; and
a seating system that is connected to a component of the frame, the seating system including a seat component;
wherein the seating system is moveable between a stored state in which the seat component is at least partially located within an expanse of the frame, and a seating position in which the seat component is spaced apart from the frame thereby permitting a user to sit on the seat component.

2. The bed of claim 1, wherein the frame includes a longitudinal support that runs along a length of the bed, the seating system comprising:
a track that is fixed to the longitudinal support;
a clamp that is slidably fixed to the track; and
a lateral bar that extends from the clamp to a seat support that supports the seat, and the connection of the lateral bar to the clamp forms a rotating joint;
wherein the seating system is moveable between the stored state and the seating position by sliding the clamp along the track and rotating the lateral bar relative to the clamp via the rotating joint.

3. (canceled)

4. The bed of claim 1, wherein:
the frame includes a longitudinal support that runs along a length of the bed and a cross support that is attached perpendicularly to the longitudinal support, the cross support further defining a hollowed recess;
the seating system comprises a rotating joint, a lateral bar, and a seat support that supports the seat, the lateral bar extending between the rotating joint at a first end and the seat support at an opposite second end; and
when the seating system in in the stored state the rotating joint and a portion of the lateral bar are located within the hollowed recess, and the seating system is moveable from the stored state to the seating position by pulling the lateral bar out from the hollowed recess until the rotating joint is outside the followed recess and rotating the lateral bar via the rotating joint.

5. The bed of claim 1, wherein the frame includes a longitudinal support that runs along a length of the bed, the seating system comprising:
a joint structure that is fixed to the longitudinal support; and
a lateral bar that extends from the joint structure to a seat support that supports the seat, and the connection of the lateral bar to the joint structure forms a rotating joint;
wherein the seating system is moveable between the stored state and the seating position by rotating the lateral bar relative to the longitudinal support via the rotating joint.

6. The bed of claim 5, wherein the joint structure comprises:
a first joint connected to the longitudinal support, the first joint permitting rotation of the lateral bar relative to the longitudinal support; and
a second joint connected to the first joint and the lateral bar, the second joint permitting a vertical movement of the lateral bar between a lower position for seating and an upper position for the stored state.

7. The bed of claim 6, wherein the lateral bar is biased in the upper position.

8. The bed of claim 5, wherein the lateral bar has a double bar configuration.

9. The bed of claim 1, wherein the frame includes a longitudinal support that runs along a length of the bed, the seating system comprising:
a lateral bar divided into segments, a segment of the lateral bar being connected to a seat support that supports the seat; and
multiple rotating joint components that connect adjacent segments of the lateral bar and that connect the lateral bar to the longitudinal support, thereby forming a double pivot mechanism;
wherein the seating system is moveable between the stored state and the seating position by rotating the lateral bar relative to the longitudinal support and rotating segments of the lateral bar relative to each other via the rotating joints.

10. The bed of claim 9, wherein the seating system comprises:
a first rotating joint that is connected to the longitudinal support;
a first lateral bar component that is connected to the first rotating joint at a first end;
a second rotating joint that is connected to a second end of the first lateral bar component; and
a second lateral bar component that is connected to the second rotating joint at a first end and the seat support at a second end;
wherein the seating system is moveable between the stored state and the seating position by rotating the first lateral bar component relative to the longitudinal support via the first rotating joint, and rotating the second lateral bar component relative to the first lateral bar component via the second rotating joint.

11-16. (canceled)

17. The bed of claim 1, wherein the frame and at least a portion of the sealing system are made of aluminum.

18. The bed of claim 1, wherein seat component is made of a moldable plastic material.

19. The bed of claim 1, wherein seat component has a saddle configuration.

20. The bed of claim 1, wherein the seating system comprises:
a bedrail attached to the frame and that includes a seat housing; and
the seat component is rotatable relative to the seat housing;
wherein in the stored state the seat component is located substantially within the seat housing, and the seating system is moveable from the stored state to the seating position by moving the seat component out from the seat housing and rotating the seat component relative to the seat housing.

21. The bed of claim 20, wherein the seat component includes a handle that extends from the seat housing when the seat portion is in the stored state.

22. The bed of claim 21, wherein the seat housing has an upper end comprising a seat ledge that supports the seat component when in the seating position and a cutaway portion that permits access to the handle when the seat component is in the stored stated.

23. The bed of any of claim 20, wherein:
the seat housing defines a seat track and the seat component includes an extension pin that is received within the seat track; and
the extension pin slides within the seat track to move the seat component out from the seat housing, and the extension pin rotates within the seat track to permit rotating the seat component relative to the seat housing.

24. The bed of claim 1, wherein the seating system comprises:
one or more bedrails attached to the frame;
a pivotal clamp that is attached to the frame; and
the seat component is connected to the pivotable clamp and is rotatable via the pivotable clamp;
wherein in the stored state the seat component is located adjacent to the one or more bedrails, and the seating system is moveable from the stored state to the seating position by moving the seat component upward relative to the frame and rotating the seat component via the pivotable clamp.

25. The bed of claim 24, wherein the pivotable clamp includes a first clamp plate, an opposing second clamp plate, and a connecting rod that extends between the clamp plates that provides a mounting interface for connection of the seat component to the pivotable clamp.

26. The bed of claim 25, wherein:
the seat component includes a seat connected to a mounting bar; and
the mounting bar includes a mounting hole that receives the connecting rod for mounting the seat component on the pivotable clamp such that the mounting bar is fixed between the first and second clamp plates.

27. The bed of claim 26, wherein the pivotable clamp further includes a lateral support plate, and an end of the mounting bar rests against the lateral support plate.

28. The seating system of claim 24, further comprising a mounting base for mounting the seating system to the frame; wherein:
the mounting base includes a slide bar upon which the pivotable clamp is mounted; and
the pivotal clamp includes a sliding support that is slidably fixed to the slide bar and that is moveable in a vertical direction to slide along the slide bar to move the seat component into and out from the stored state.

29. (canceled)

30. The bed of claim 1, wherein the bed portion includes a head portion, and when the seating system is in the seating position the seat component is located at a distance between 38 and 48 inches from the head portion.
